(19) United States
(12) Patent Application Publication
(10) Pub. No.: US 2004/0215072 A1
(43) Pub. Date: Oct. 28, 2004

(54) Method of medical imaging using combined near infrared diffusive light and ultrasound

(76) Inventor: Quing Zhu, Mansfield Center, CT (US)

Correspondence Address: CANTOR COLBURN, LLP, 55 GRIFFIN ROAD SOUTH, BLOOMFIELD, CT, 06002

(21) Appl. No.: 10/764,268

(22) Filed: Jan. 23, 2004

Related U.S. Application Data
(60) Provisional application No. 60/442,528, filed on Jan. 24, 2003.

(51) Int. Cl. A61B 6/00; A61B 8/00

(52) U.S. Cl. 600/407; 600/443; 600/473

(57) ABSTRACT

An image reconstruction process using a combined near infrared and ultrasound technique and its utility in imaging distributions of optical absorption and hemoglobin concentration of lesions is described. In the image reconstruction process, the tissue volume is segmented, based on initial co-registered ultrasound measurements, into a lesion region and a background region. Reconstruction is performed using a finer grid for the lesion region and a relatively coarser grid for the background tissue. In one embodiment, image reconstruction is refined by optimizing lesion parameters measured from ultrasound images.

### CROSS REFERENCE TO RELATED APPLICATIONS

This application claims the benefit of U.S. Provisional Application Serial No. 60/442,528, filed Jan. 24, 2003.

### BACKGROUND OF THE INVENTION

This invention relates primarily to the field of medical imaging and, more specifically, to a method of medical imaging using combined near infrared diffusive light and ultrasound.

Ultrasound imaging is a well-developed medical diagnostic that is used extensively for differentiation of cysts from solid lesions in breast examinations, and it is routinely used in conjunction with mammography to differentiate simple cysts from solid lesions. Ultrasound can detect breast lesions that are a few millimeters in size; however, its specificity in breast cancer detection is not high as a result of the overlapping characteristics of benign and malignant lesions. The sonography appearance of benign and malignant lesions have considerable overlapping features, which has prompted many radiologists to recommend biopsies on most solid nodules. Thus, the insufficient specificity provided by ultrasound results in a large number of biopsies yielding benign breast masses or benign breast tissue (currently 70 to 80 percent of biopsies yield benign changes).

Optical diagnostics based on diffusing near infrared (NIR) light have also been employed in breast cancer detection. Functional imaging with NIR light is made possible in a spectrum window that exists within tissues in the 700-900 nanometer (run) NIR region, in which photon transport is dominated by scattering rather than absorption. Functional imaging with NIR light offers several tissue parameters to differentiate tumors from normal breast tissue.

It has been shown that breast cancers have higher blood volumes than non-malignant tissue due to angiogenesis, especially at the cancer periphery. Tumor blood volume and micro-vascular density are parameters anatomically and functionally associated with tumor angiogenesis. During the last decade, modeling of the light propagation in the near infrared (NIR) region, combined with the advancements of light source and detectors, has improved the diffused light measurements and made possible the application of tomographic techniques for characterizing and imaging tumor angiogenesis. However, the NIR technique has not been widely used in clinics and the fundamental problem remains the intense light scattering. As a result, diffusive light probes a widespread region instead of providing information along a straight line, and tomographic image reconstruction using NIR is, in general, underdetermined and ill-posed.

### BRIEF DESCRIPTION OF THE INVENTION

The above-described drawbacks and deficiencies are overcome or alleviated by a method for imaging a lesion using combined near infrared diffusive light and ultrasound, the method comprising: scanning a subject with ultrasound waves to obtain ultrasound images of a scanned volume, the scanned volume including the lesion; scanning the subject with near infrared light to obtain optical measurements of the scanned volume; segmenting the scanned volume into a lesion region including the lesion and a background region absent the lesion using the ultrasound images; and reconstructing from the optical measurements an optical image of at least a portion of the scanned volume, the reconstructing being performed using different voxel sizes for optical measurements corresponding to the lesion region and optical measurements corresponding to the background region. The optical image may indicate at least one of wavelength-dependent absorption associated with the lesion and hemoglobin concentration associated with the lesion.

In another aspect, a method for imaging a lesion using combined near infrared diffusive light and ultrasound includes: measuring parameters of the lesion using the ultrasound images to provide values indicative of the parameters; and reconstructing the optical image again using the values.

### BRIEF DESCRIPTION OF THE DRAWINGS

FIG. 1 is a simplified block diagram of a combined ultrasound and NIR light imaging system;

FIG. 2 is a diagram of a combined ultrasound and NIR light probe for use with the system of FIG. 1;

FIG. 3 is a simplified block diagram of an NIR light imaging system;

FIG. 4 is a flowchart depicting a method for imaging a lesion using combined near infrared diffusive light and ultrasound;

FIG. 5 is a flowchart depicting an alternative embodiment of a method for imaging a lesion using combined near infrared diffusive light and ultrasound;

FIG. 6 is an ultrasound image of a first lesion;

FIG. 7 is a series of reconstructed optical images indicating wavelength-dependent absorption associated with the first lesion, the images being obtained using a 780 nm wavelength laser diode;

FIG. 8 is a series of reconstructed optical images indicating wavelength-dependent absorption associated with the first lesion, the images being obtained using an 830 nm wavelength laser diode;

FIG. 9 is a series of reconstructed optical images indicating hemoglobin concentration associated with the first lesion;

FIG. 10 is an ultrasound image of a second lesion;

FIG. 11 is a series of reconstructed optical images indicating wavelength-dependent absorption associated with the second lesion, the images being obtained using a 780 nm wavelength laser diode;

FIG. 12 is a series of reconstructed optical images indicating wavelength-dependent absorption associated with the second lesion, the images being obtained using an 830 nm wavelength laser diode;

FIG. 13 is a series of reconstructed optical images indicating hemoglobin concentration associated with the second lesion;

FIG. 14(a) represents the image of a large 4 centimeter (cm)×4 cm×1.5 cm palpable mass, located at the 6 to 8 o'clock position of the left breast that was considered to be highly suspicious for malignancy;

FIGS. 14(b) and (c) represents optical absorption maps at wavelengths of 780 and 830 nm respectively and show that the distributions are highly heterogeneous with high absorption at the tumor periphery;

FIG. 14(d) depicts a series of optical images showing the hemoglobin concentrations;

FIG. 15(a) is an ultrasound image of the cancer three month later;

FIG. 15(b) and (c) are optical absorption maps taken three months later at wavelengths of 780 and 830 nm;

FIG. 15(d) is the total hemoglobin distribution;

FIG. 15(e) is a photograph showing a representative section having a high microvessel density;

FIG. 16(a) is an ultrasound image showing a hypoechoic mass having a size of 3 cm×3 cm×2 cm at the 2 o'clock position in the left breast;

FIG. 16(b) and (c) are optical absorption maps obtained at 780 nm and 830 nm wavelengths respectively;

FIG. 16(d) represents the total hemoglobin distribution;

FIG. 16(e) is a photograph showing a representative section having a high microvessel density;

FIG. 17(a) is an ultrasound image showing the lesion;

FIGS. 17(b) and (c) depicts a series of optical absorption maps and total hemoglobin concentration maps obtained 8 days after the core biopsy;

FIG. 17(d) represents the total hemoglobin distribution;

FIG. 18(a) is an ultrasound image showing the lesion;

FIGS. 18(b) and (c) depicts a series of optical absorption maps and total hemoglobin concentration maps; and

FIG. 18(d) represents the total hemoglobin distribution.

### DETAILED DESCRIPTION OF THE INVENTION

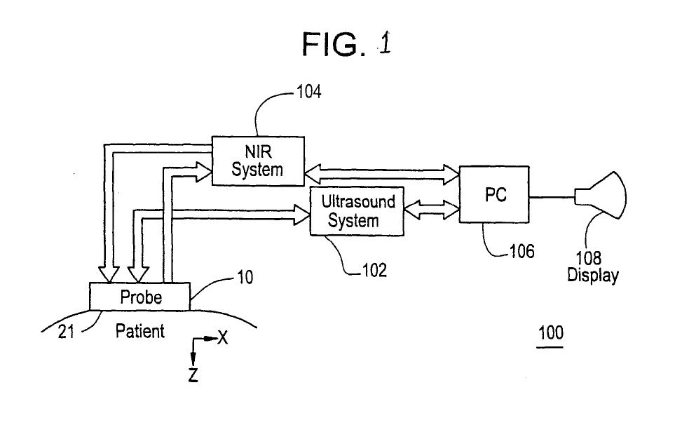

Referring now to FIG. 1, a simplified block diagram of a combined ultrasound and NIR light imaging system is shown generally at 100. In the embodiment shown, the combined system 100 includes a combined ultrasound and NIR light probe 10, which is operatively connected to an ultrasound imaging system 102 and an NIR light imaging system 104. While a combined ultrasound and NIR light probe 10 is shown, the present invention may also be implemented with separate ultrasound and NIR light probes. The ultrasound and NIR imaging systems 102, 104 provide output to an associated computer (PC) 106, which, in turn, provides output to a display device 108, such as a cathode ray tube (CRT) or a printer. The PC 106 is programmed to perform various beamforming and signal processing algorithms. The PC 106 is further programmed to perform a method for imaging tumor angiogenesis using combined near infrared diffusive light and ultrasound.

FIG. 2 shows the front face of the combined ultrasound and NIR light probe 10. The combined probe 10 includes a one-dimensional ultrasound array 12 located at the center of the probe 10 and coupled to the ultrasound imaging system 102. The combined probe 10 also includes NIR light source and detection elements 16, 18 distributed at the periphery of the ultrasound array 12 and coupled to the NIR imaging system 104. The ultrasound array 12 and NIR light source and detection elements 16, 18 may be mounted on a common probe support 20. Preferably, combined probe 10 is sized to be hand-held. In this embodiment, the probe support 20 is made of a plastic plate 10 centimeter (cm) in diameter. The probe support 20 has a substantially flat face surface 21, which is directed towards the patient or target of interest.

The ultrasound array 12 may be any commercially available ultrasound probe. In the present embodiment, a one-dimensional array is used. It will be recognized, however, that any ultrasound array may be incorporated in the combined probe. For example, two-dimensional, 1.5-dimensional, or 1.75-dimensional ultrasound array can be used. It is further contemplated that the ultrasound array 12 can be releasably secured to the probe 10 to allow independent ultrasound or NIR imaging if needed.

The NIR light source and detection elements 16, 18 include twelve source elements 16 and eight detector elements 18. In the embodiment shown, sources 16 are formed from optical fibers (source fibers) connected to a laser diode source (not shown). Each detector 18 is formed from an optical fiber (detection fiber) connected to a light detector (not shown). The source fibers and detector fibers extend through support 20, with the ends of each fiber being substantially flush with face surface 21. The source elements 16 are arranged in a two-by-six grid pattern on one side of the ultrasound array 12, and the detection elements 18 are arranged in a triangular pattern on the opposite side of ultrasound array 12. While source elements 16 are shown as fibers mounted to support 20, source elements 16 may alternatively include laser diode sources connected directly to probe 10, without the source fibers. Similarly, detector elements 18 may include light detectors directly connected to probe 10, without the detector fibers.

Referring again to FIG. 1, any NIR light imaging system 104 and ultrasound imaging system 102 may be used to implement the present invention. For example, a commercially available ultrasound imaging system can be used for ultrasound imaging system 102. As another example, the NIR imaging system described by R. M. Danen, Y. Wang, X. D. Li, W. S. Thayer, and A. G. Yodh (1998), in their paper entitled “Regional Imager for Low Resolution Functional Imaging of the Brain with Diffusing Near-infrared Light,” Photochemistry and Photobiology, January 1998, vol 67, can be applied as NIR imaging system 104. In another example, a combined ultrasound and NIR imaging system as described in U.S. Pat. No. 6,264,610 may be employed.

For the ultrasound imaging system 102, a data acquisition cycle starts with a transmit period in which one or more elements in ultrasound array 12 are excited with signals of various delays and amplitudes according to various transmit beamforming algorithms. After the transmit period, one or more elements in the ultrasound array 12 begin to receive the ultrasound echoes from various discontinuities in the medium (e.g. the patient) and to transform them into electrical signals. The received electrical signals are then amplified and multiplexed to produce a series of amplified signals. The series of amplified signals is then provided to the PC 106, where the signals are processed in various ways, e.g. amplified, filtered, beamformed, detected, and eventually transformed into a set of digital values (pixels) that can be displayed on the display device 108 (FIG. 4).

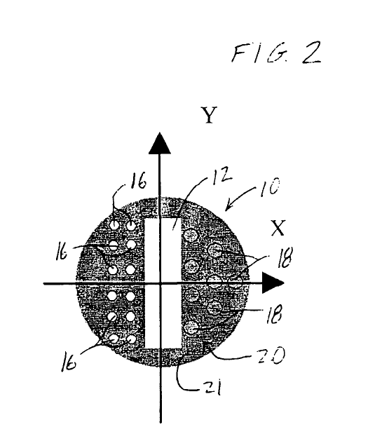

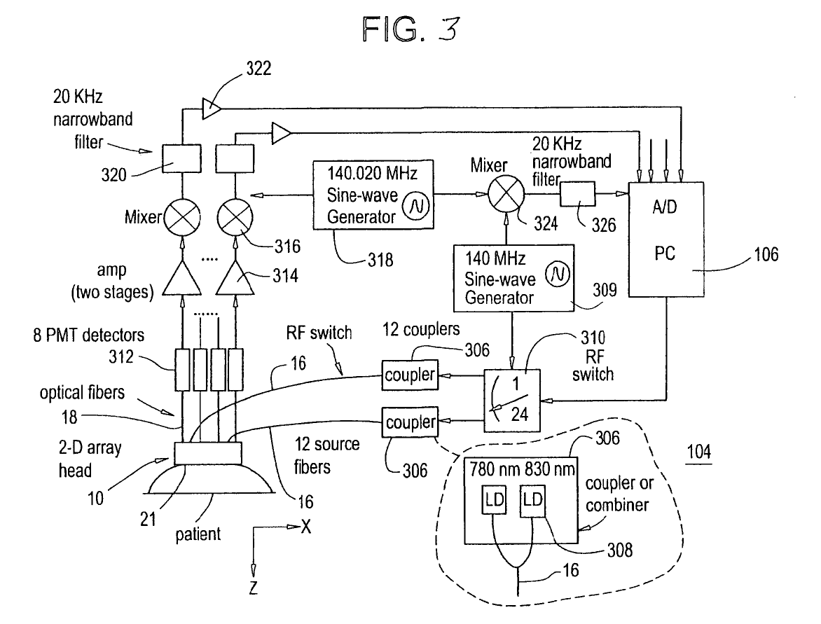

Referring to FIG. 3, a block diagram of one example of an NIR imaging system 104 is shown. The NIR light imaging system 104 includes twelve dual wavelength source channels and eight parallel receiving channels. On the source side of NIR imaging system 104, twelve optical couplers or combiners 306 each house dual wavelength (780 nm and 830 nm) laser diodes 308, as shown in the insert portion of FIG. 3. Optical couplers 306 include, for example, those manufactured by OZ optics, Inc. The output of laser diodes 308 are coupled to the transducer probe 10 through twelve, multi-mode optic fibers, to form NIR sources 16. Each laser diode 308 has its own driving circuit (not shown here) and its output intensity is modulated at a predetermined frequency (e.g., 140 MHz) by a local oscillator (sine wave generator) 309. The input of each laser diode 308 is coupled to a corresponding RF source switch 310. The twenty-four RF source switches 310 are controlled in series by the PC 106 to direct the output of the oscillator 309 to the laser diodes 308 corresponding to a single wavelength (780 nm or 830 nm). The laser diodes 308 corresponding to the selected wavelength then provide photon diffusion waves at the selected wavelength to the NIR source fibers, which project the photon diffusion waves into the medium (e.g. the patient).

On the reception side of NIR imaging system 104, optical fibers receive reflected photon diffusion waves from targets in the medium (e.g. the patient) and guide the reflected waves to the input of a corresponding photon multiplier tube (PMT) detector 312. The parallel outputs of the eight PMT detectors 312 are amplified (e.g., by 40 dB) by amplifiers 314 and mixed with an output signal at a predetermined frequency (e.g., 140.02 MHz) from a local oscillator 318 by mixers 316. The heterodyned signals output by mixers 316 are filtered by narrowband filters 320 and further amplified (e.g., by 30 dB) by amplifiers 322. The amplified signals are then sampled at a predetermined frequency (e.g., 250 KHz) by an analog to digital conversion (A/D) board inside the PC 106. The signals output by oscillators 309 and 318 respectively, are directly mixed by mixer 324 to produce reference signal (e.g., a 20 kHz reference signal). The 20 kHz reference signal is filtered by a 20 kHz narrow band filter 326, and provided as input to the PC 106.

PC 106 is programmed to perform a Hilbert transform on both sampled and reference waveforms. The amplitude of the Hilbert transform of the sampled waveform corresponds to the measured amplitude, and the phase difference between the phases of Hilbert transforms of sampled and reference waveforms correspond to the measured phase. Both amplitude and phase at each source-detector pair are obtained and the resulting total number of measurements is equal to twice the product of the number of laser diodes and the number of receiving channels (e.g., 12×8×2=192). Measurements made at the multiple source-detector positions can be used in the following image reconstruction process to reconstruct an image of the tissue volume at slice depths below the probe 10.

Referring to FIG. 4, an image reconstruction process 400 is shown. Image reconstruction process 400 may be used where the lesion size is small or where the lesion is well-defined. The image reconstruction process may be performed using the co-registered (commonly centered) ultrasound images and optical measurements obtained using combined ultrasound and NIR light imaging system 100 (blocks 402, 404). In the image reconstruction process, parameters of the lesion are measured from the ultrasound image (block 406). The parameters of the lesion may include lesion location within the volume scanned and the size of the lesion. Using the estimated lesion parameters, the entire tissue volume is then segmented into a lesion region, L, and a background region, B (block 408). Reconstruction is then performed using a finer grid for lesion region L and a relatively coarser grid for the background region B (block 410). As a result, the total number of voxels with unknown absorption can be maintained on the same order of total measurements and the matrix with unknown total absorption distribution is appropriately scaled for inversion. Detailed distributions of wavelength-dependent absorption and hemoglobin concentration of the lesion can be obtained using the reconstruction process 400.

In the present embodiment, probe support 20 is made of a circular black plastic plate of a known diameter (e.g., 10 cm), therefore, a semi-infinite boundary condition can be used for NIR measurement geometry. The measured amplitude Â_{αβ} and phase \hat{φ}_{αβ} after calibration of the NIR light imaging system 104 follow simple equations as

log(ρ_{αβ}²Â_{αβ})=−kᵢρ_{αβ}+C₁, \hat{φ}_{αβ}=kᵣρ_{αβ}+C₂ (1)

where α and β are source and detector channels, respectively, ρ_{αβ} is the corresponding source-detector separation, k=kᵢ+jkᵣ is the wavenumber, and C1 and C2 are constants. By fitting Â_{αβ} and \hat{φ}_{αβ} measured from normal tissue (absent of lesions) kᵣ and kᵢ can now be obtained from the slopes of equation (1), and background absorption \overline{μ}ₐ and reduced scattering coefficient \overline{μ}′ₛ can be calculated as

μₐ=−\overline{D}(kᵣ²−kᵢ²), \overline{μ}′ₛ=1/(3\overline{D}) with \overline{D}=ω/(2νkᵣkᵢ) (2)

In the image reconstruction process 400, the entire tissue volume is segmented based on initial co-registered ultrasound measurements into a lesion region, L, and a background region, B (block 408). A Born approximation may then be used to relate the scattered field U′_{sc} (rₛᵢ, r_{di}, ω) measured at the source-detector pair i to absorption variations Δμₐ (r′) in each volume element of two regions within the sample

$${U}_{sc}^{′} ( {r}_{si} , {r}_{di} , ω ) = - \frac{1}{\overline{D}} ( {\int }_{L}^{} G ( {r}^{′} , {r}_{di} ) {U}_{inc} ( {r}^{′} , {r}_{si} ) Δ {μ}_{a} ( {r}^{′} ) {ⅆ}^{3} {r}^{′} + {\int }_{B}^{} G ( {r}^{′} , {r}_{di} ) {U}_{inc} ( {r}^{′} , {r}_{si} ) Δ {μ}_{a} ( {r}^{′} ) {ⅆ}^{3} {r}^{′} ) \qquad ( 2 )$$

where U_{inc}(r′, rₛᵢ, ω) and G(r′, r_{di}, ω) are incident wave and green function of semi-infinite geometry, respectively; and rₛᵢ and r_{di} are source and detector positions. The lesion region L and background region B are then discretized with different voxel sizes (a finer grid for lesion region L and a relatively coarser grid for background region B). The scattered field can then be approximated as

$${U}_{sc}^{′} ( {r}_{si} , {r}_{di} , ω ) \approx - \frac{1}{\overline{D}} ( {\sum }_{{L}_{j}}^{} G ( {r}_{vj} , {r}_{di} ) {U}_{inc} ( {r}_{vj} , {r}_{si} ) {\int }_{j}^{} Δ {μ}_{a} ( {r}^{′} ) {ⅆ}^{3} {r}^{′} + {{\sum }_{{B}_{k}}^{}}^{} G ( {r}_{vk} , {r}_{di} ) {U}_{inc} ( {r}_{vk} , {r}_{si} ) {\int }_{k}^{} Δ {μ}_{a} ( {r}^{′} ) {ⅆ}^{3} {r}^{′} ) \qquad ( 3 )$$

where rᵥⱼ and rᵥₖ are centers of voxel j and k in lesion region L and background region B, respectively. The matrix form of equation (3) is given as

[U_{sd}]_{M×1}=[W_{L}, W_{B}]_{M×N}[M_{L}, M_{B}]^{T},

where

$${W}_{L} = {[ - \frac{1}{D} G ( {r}_{vj} , {r}_{di} ) {U}_{inc} ( {r}_{vj} , {r}_{si} ) ]}_{M \times {N}_{L}}$$

and

$${W}_{B} = {[ - \frac{1}{D} G ( {r}_{vk} , {r}_{di} ) {U}_{inc} ( {r}_{vk} , {r}_{si} ) ]}_{M \times {N}_{B}}$$

are weight matrixes for lesion and background regions, respectively;

$$[ {M}_{L} ] = [ {\int }_{{1}_{L}}^{} {Δμ}_{a} ( {r}^{′} ) {ⅆ}^{3} {r}^{′} , … {\int }_{{N}_{L}}^{} {Δμ}_{a} ( {r}^{′} ) {ⅆ}^{3} {r}^{′} ]$$

and

$$[ {M}_{B} ] = [ {\int }_{{1}_{B}}^{} {Δμ}_{a} ( {r}^{′} ) {ⅆ}^{3} {r}^{′} , … {\int }_{{N}_{B}}^{} {Δμ}_{a} ( {r}^{′} ) {ⅆ}^{3} {r}^{′} ]$$

are total absorption distributions of lesion and background regions, respectively.

Instead of reconstructing Δμₐ distribution directly, as is done in the standard Born approximation, the total absorption distribution M is reconstructed and then the total is divided by different voxel sizes of lesion and background tissue to obtain the Δμₐ distribution. By choosing a finer grid for lesion and a relatively coarser grid for background tissue, we can maintain the total number of voxels with unknown absorption on the same scale of the total measurements. As a result, the inverse problem is less underdetermined. In addition, since the lesion absorption coefficient is higher than that of background tissue, in general, the total absorption of the lesion over a smaller voxel is on the same scale of total absorption of the background over a bigger voxel, therefore the matrix [M_{L}, M_{B}] is appropriately scaled for inversion. The reconstruction is formulated as least square problem and the unknown distribution M can be iteratively calculated using conjugate gradient method (block 410). The Δμₐ distributions of lesion and background are readily calculated from the total absorption distribution M by dividing M with different voxel sizes.

Referring to FIG. 5, a two-part image reconstruction process 500 is shown. The two-part image reconstruction process 500 may be used for larger, less-defined lesions to produce detailed distributions of wavelength-dependent absorption and hemoglobin concentration of the lesion. The two-part reconstruction process may be performed using the co-registered ultrasound images and optical measurements obtained using combined ultrasound and NIR light imaging system 100 (blocks 402, 404). The first-part of the image reconstruction process 500 is substantially similar to image reconstruction process 400, as described with reference to FIG. 4. In the second part, indicated at 502, the lesion parameters are refined by increasing the lesion size (block 504) to account for possible inaccuracies of the initial lesion size estimate in block 406. The entire tissue volume is again segmented based the refined ultrasound measurements into a lesion region, L, and a background region, B (block 506), and reconstruction is performed using a finer grid for lesion region L and a relatively coarser grid for the background region B (block 508). The reconstruction performed in block 508 may be substantially similar to that used in block 410.

Because a finer grid is used for the lesion region L (block 508), increasing the lesion size parameter (block 504) will increase the number of voxels in the optical image. If the number of voxels is increased well above the total number of measurements, the optical image may be unobtainable. For this reason, the increase in the lesion size (block 504) may be limited by the number of voxels that will be produced using the increased lesion size, and it may be necessary to increase the lesion size (block 504) while controlling the number of voxels.

Referring to FIGS. 4 and 5, determination of the lesion parameters (blocks 406 and 504) may be accomplished as follows. The commercial 1-D (one dimensional) ultrasound probe acquires 2-D (two dimensional) ultrasound images in y-z plane (z is the propagation direction) and the 2-D NIR probe provides 3-D (three dimensional) optical measurements for 3-D image reconstruction. Therefore, at each location, a 2-D ultrasound image is co-registered with a corresponding set of 3-D optical measurements in y-z plane. To estimate the size of the lesion using 2-D ultrasound images, the lesion is approximated as an ellipsoid, and its diameters are estimated from two orthogonal ultrasound images. If the lesion is small, the 3-D lesion center can be estimated accurately from two orthogonal 2-D ultrasound images. Thus, the image reconstruction process 400 will produce accurate results for small lesions. However, if the lesion is large and irregular, it may be difficult to estimate the lesion center from two orthogonal 2-D ultrasound images. In addition, the diameter measurements of large, irregular lesions may be inaccurate because lesion boundaries may not be well defined in ultrasound images. Furthermore, the target boundaries seen by different modalities may be different due to different contrast mechanisms. To overcome these limitations associated with large, irregular lesions, image reconstruction process 500 includes an additional refinement of the lesion parameters (block 504), where the lesion center is estimated from the 2-D co-registered ultrasound image and diameters in one or more spatial dimensions are estimated to be larger than the diameters measured in the ultrasound images. The diameters may be increased such that the lesion size parameter is sufficient to include suspicious areas outside the lesion. As discussed above, the lesion size parameter may be limited by the number of voxels. The lesion size parameter may also be limited by the size of the probe (e.g., 10 cm in diameter). This procedure accounts for errors in center and diameter measurements obtained from the co-registered ultrasound images. The lesion depth z and lesion boundaries in z direction can be estimated reasonably well from 2-D co-registered ultrasound. We have found from experiments that the measurement inaccuracies of lesion spatial location and size have little affect on reconstructed optical properties as long as the lesion depth is measured accurately and the total unknown image voxel number is controlled at the same order as the total measurements.

### EXAMPLES

### Example 1

Clinical studies were performed using the above-described reconstruction method. Patients with palpable and non-palpable masses that were visible on clinical ultrasound imaging systems were used as subjects. These subjects were scanned with the combined probe, and ultrasound images and optical measurements were acquired at multiple locations including the lesion region scanned at two orthogonal positions and a normal region of the contralateral breast scanned at two orthogonal positions. FIG. 6 shows a gray scale ultrasound image of a palpable lump. The lesion was located in a breast of a human patient at approximately 1.5 cm depth. Ultrasound showed an irregular poorly defined hypoechoic mass and the lesion was considered as highly suspicious for malignancy. An ultrasound guided core needle biopsy revealed that the lesion was a high grade in-situ ductal carcinoma with necrosis.

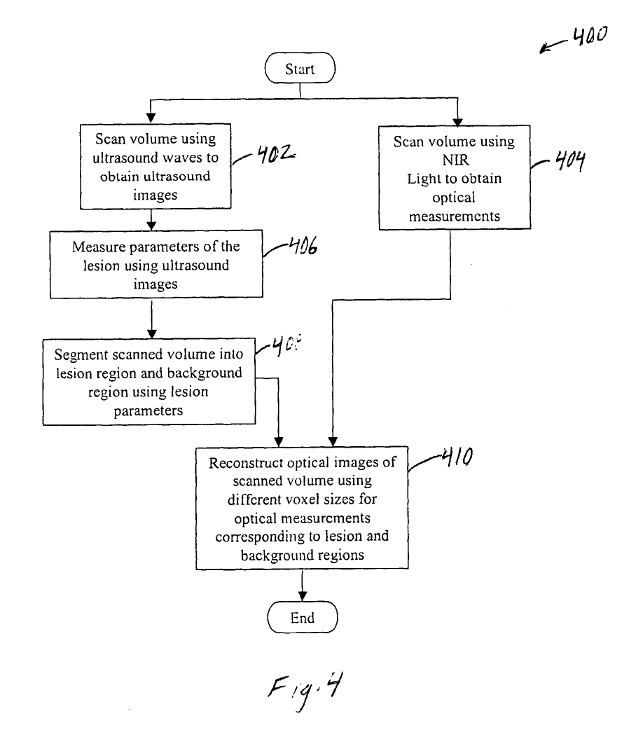

Multiple optical measurements at two orthogonal positions were simultaneously made with ultrasound images at the lesion location as well as at approximately the same location of the contralateral normal breast. The fitted average tissue background values measured at normal side of the breast were \overline{μ}⁷⁸⁰=0.03 cm⁻¹, \overline{μ}ₐ⁸³⁰=0.005 cm₋₁, μ′ₛ⁷⁸⁰=9.22 cm⁻¹, μ′ₛ⁸³⁰=7.61 cm⁻¹. The perturbations for both wavelengths used to calculate absorption maps were normalized as

$${U}_{sc}^{′} ( {r}_{si} , {r}_{di} , ω ) = \frac{{U}_{L} ( {r}_{si} , {r}_{di} , ω ) - {U}_{N} ( {r}_{si} , {r}_{di} , ω )}{{U}_{N} ( {r}_{si} , {r}_{di} , ω )} {U}_{B} ( {r}_{si} , {r}_{di} , ω ) ,$$

where U_{L}(rₛᵢ, r_{di}, ω) and U_{N}(rₛᵢ, r_{di}, ω) were measurements obtained at lesion region and contralateral normal region, and U_{B}(rₛᵢ, r_{di}, ω) was calculated incident field using fitted background. This procedure cancels unknown system gains associated with different sources and detectors as well as electronic channels. The initial estimate of lesion center and diameters from ultrasound images were (0, 0.39, 1.7) cm and 3.44×4.38×1.76 cm, respectively. A finer grid of 0.5×0.5×0.5 (cm3) and a relatively coarser grid of 1.5×1.5×1.0 (cm3) were chosen for the lesion and background tissue, respectively. The total reconstruction volume was chosen to be 9×9×4 cm3 and the total number of voxels with unknown optical absorption was 190, which was on the same order of 192 total measurements. The image reconstruction was performed using the NIR data simultaneously acquired with the ultrasound image shown in FIG. 7. The second step, refined reconstruction, revealed optimal diameters of 4.28×5.18×1.96 cm of the lesion. The detailed absorption maps with high absorption non-uniformly distributed around the lesion boundaries at both wavelengths are shown in FIGS. 8 and 9. By assuming that the major chromophores are oxygenated (oxyHb) and deoxygenated (deoxyHb) hemoglobin molecules in the wavelength range studied, we can estimate the distribution of total hemoglobin concentration as shown in FIG. 9. The extinction coefficients used for calculating oxyHb and deoxyHb concentrations were

$${ϵ}_{Hb}^{780} = 2.544 , {ϵ}_{{HbO}_{2}}^{780} = 1.695 , {ϵ}_{Hb}^{830} = 1.797 , {ϵ}_{{HbO}_{2}}^{830} = 2.419$$

in a natural logarithm scale with units of inverse millimoles times inverse centimeters. The measured average cancer and background total hemoglobin concentrations were 50.83 μ moles and 20.70 μ moles, respectively.

The absorption distributions at both wavelengths as well as total hemoglobin concentration were distributed heterogeneously at the cancer periphery. Such fine distributions have not been reported by using NIR only reconstruction techniques and they are valuable for breast cancer diagnosis and treatment. This finding agrees with the published literature showing that breast cancers have higher blood volumes than non-malignant tissue due to angiogenesis, especially at the cancer periphery. In addition, the carcinoma reported here had a necrotic core which could lead to low absorptions observed at both wavelengths in the center region.

### Example 2

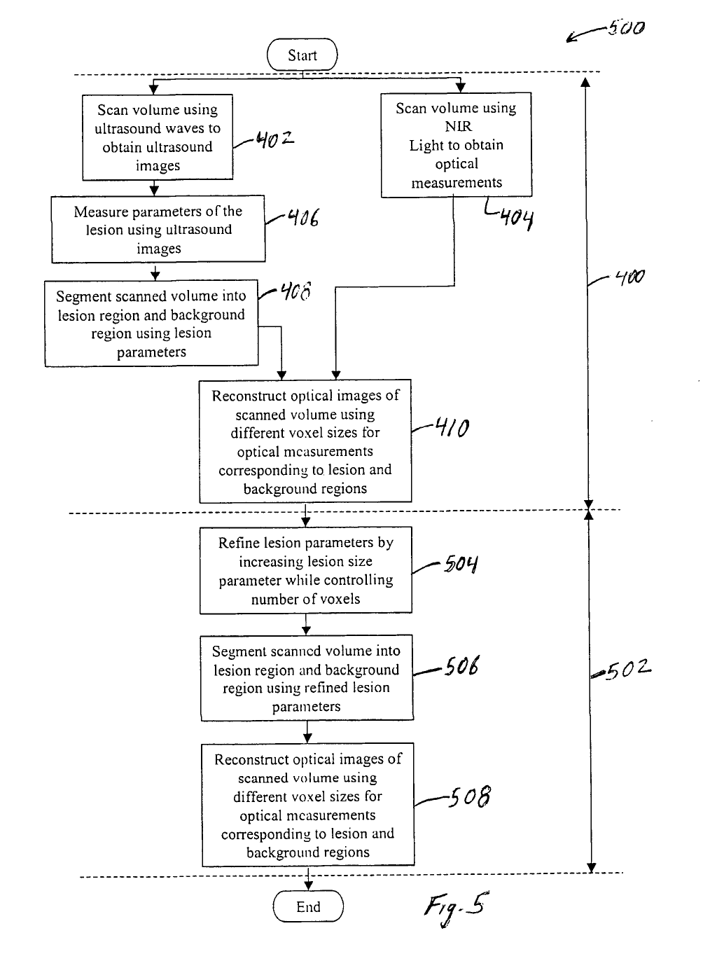

Another example, as shown in FIGS. 10-13, was obtained from a 56-year-old woman who had a non-palpable lesion located at the 10 o'clock position of the left breast. An ultrasound image, shown in FIG. 10, showed a solid mass with internal echoes measuring 9 mm in size and the lesion was considered suspicious. An ultrasound guided core needle biopsy was recommended and biopsy results revealed that the lesion was in-situ and invasive ductual carcinoma with ductual and lobular features (nuclear grade II, histological grade II). The tumor removed from the breast measures 1.5 cm in greater diameter and is composed predominantly of invasive carcinoma (>80%) extending to inferior/anterior surgical margin.

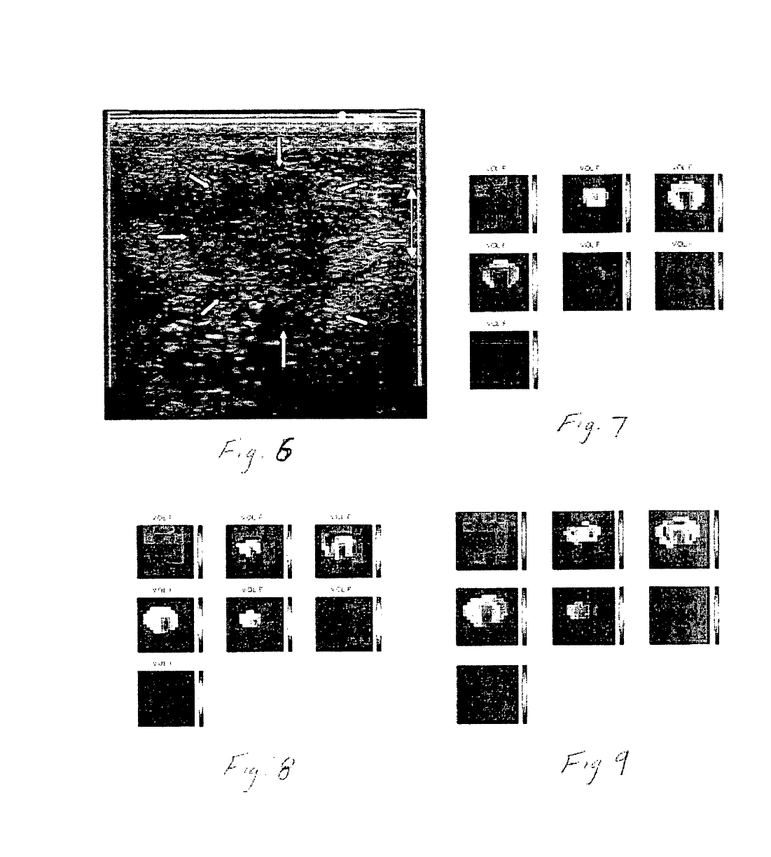

The fitted average tissue background absorption coefficient μₐ and reduced scattering coefficient μ′ₛ at 780 nm and 830 nm were measured as \overline{μ}ₐ⁷⁸⁰=0.035, respectively. The initial estimate of the lesion center and diameters measured by co-registered ultrasound were (0, −0.56, 1.9) cm and 9 mm, respectively. A 6 cm diameter was used in both x and y spatial dimensions at the center of (0, −0.56, 1.9) cm for finer optical reconstruction. The same fine and coarse voxel sizes as in the previous example were used and the total unknown voxels was 256. The optical absorption maps at both wavelengths are shown in FIG. 11 and 12, respectively. In FIGS. 11 and 12 the first slice is 0.4 cm deep into the breast tissue from the skin surface and the last slice is closer to the chest wall. The spacing between the slices is 0.5 cm. This lesion has shown much larger spatial extent at 780 nm than that at 830 nm. The measured maximum absorption coefficients are μₐ⁷⁸⁰=0.28 cm-1 and μₐ⁸³⁰=0.24 cm-1, respectively. The absorption maximums at both wavelengths are located at (0 −1.0 1.9) cm, which is close to the lesion center as measured using ultrasound images. The geometry mean of the optical mass at both wavelengths are measured as 2 cm, which is two times larger than the 9 mm diameter measured by ultrasound. This suggests that optical contrasts extend well beyond the cancer periphery due to angiogenesis. FIG. 13 is the distribution of total hemoglobin concentration. The measured maximum and average total hemoglobin concentration of the lesion are 122.68 moles and 87.5 moles, respectively, and the measured background hemoglobin concentration is 21.4 moles.

The absorption distributions at both wavelengths as well as total hemoglobin concentration were resolved well for such a small (9 mm) lesion. Such resolution is unattainable using optical only reconstruction.

### Example 3

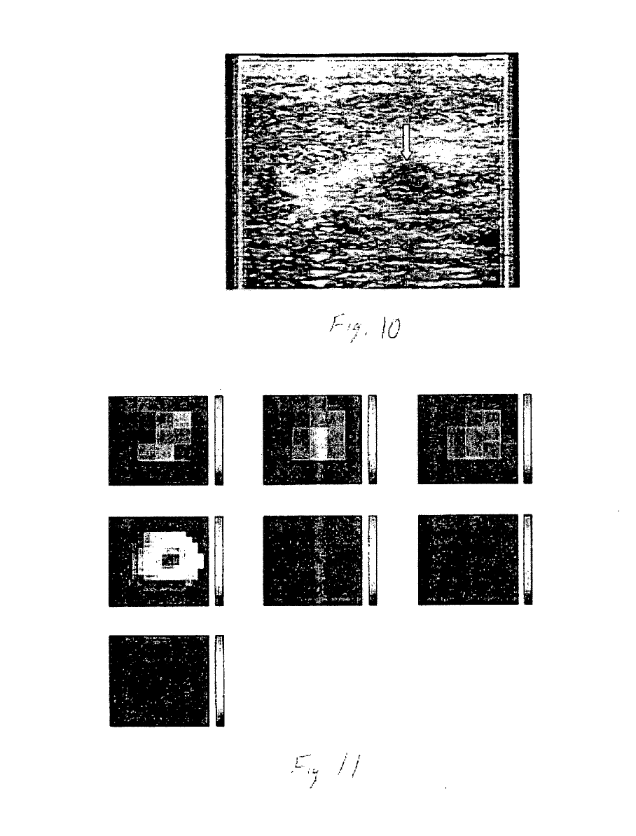

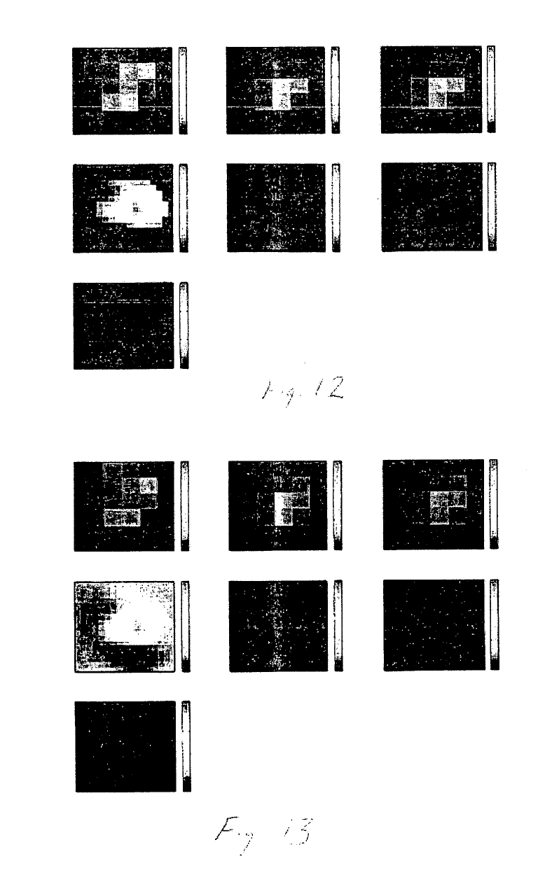

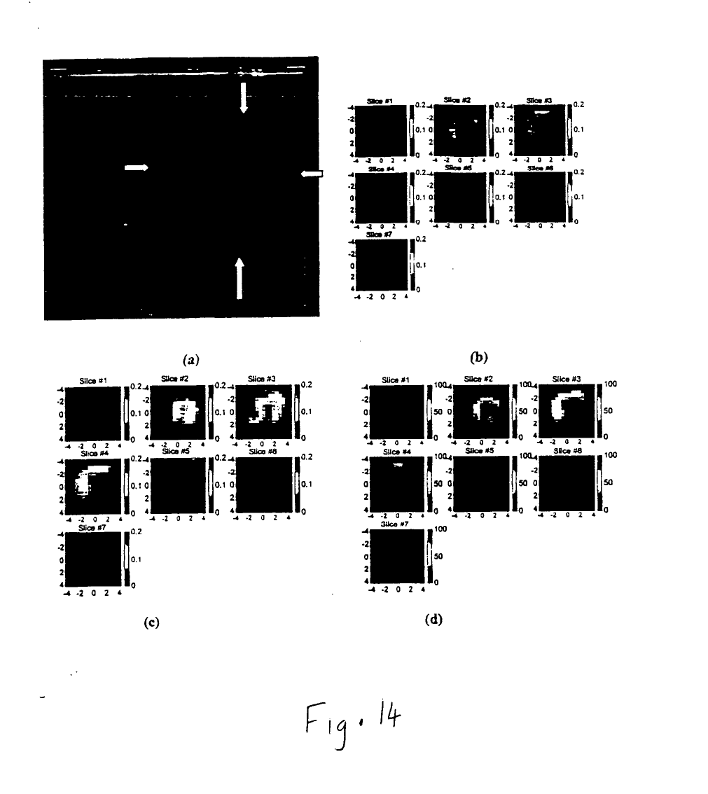

During this diagnostic imaging study, a patient who was undergoing chemotherapy was scanned. The patient, a 44-year-old woman had a large 4 cm×4 cm×1.5 cm palpable mass, shown in FIG. 14(a), located at the 6 to 8 o'clock position of the left breast that was considered to be highly suspicious for malignancy. The lesion center was approximately 1.5 cm in depth relative to the skin. An ultrasound guided needle biopsy revealed that the lesion was a high-grade invasive carcinoma with necrosis. Optical absorption maps of both wavelengths are shown in FIGS. 14(b) and (c) and the distributions are highly heterogeneous with high absorption at the cancer periphery. To account for possible larger spatial extension of cancer optical contrast, the region of interest used for finer grid optical reconstruction was chosen as 8.4 cm×8.4 cm×1.9 cm, which was much larger than the region measured by ultrasound. Slice 1 is the spatial x-y image of 9 cm×9 cm obtained at 0.5 cm deep from the skin surface. Slice 7 is 3.5 cm deep toward the chest wall and the spacing between slices is 0.5 cm. The vertical scale is the absorption coefficient in cm⁻¹ ranging from 0 to 0.2 cm⁻¹. The total hemoglobin concentration map is shown in FIG. 14(d) and the maximum and average hemoglobin concentrations are 92.1 μmol/liter and 26.2 μmol/liter, respectively. The vertical scale of FIG. 14(d) is in μmol/liter ranging from 0 to 100 μmol/liter. The measured maximum absorption coefficients at 780 nm and 830 nm were 0.17 cm⁻¹ and 0.22 cm⁻¹, respectively. Since this cancer was too large for breast conserving surgery, the patient was treated with chemotherapy in the neo-adjuvant setting for three months. At the time, the patient completed the chemotherapy, she was imaged again with the ultrasound and the near-infrared probe. FIG. 15(a) is the ultrasound image of the cancer three month later. The cancer contrast is poor and cancer boundaries are completely unclear, probably due to treatment. FIG. 15(b) and (c) are optical absorption maps at both wavelengths and (d) is the total hemoglobin distribution. The same ROI for finer grid optical reconstruction as used in FIG. 14 was chosen. The maximum and average hemoglobin concentrations of the lesion were 79.8 μmol/liter and 24.9 μmol/liter, respectively. The measured maximum absorption coefficients at 780 nm and 830 nm were 0.15 cm⁻¹ and 0.19 cm⁻¹, respectively. Compared with the images acquired before treatment, the spatial extension of the light absorption patterns was much smaller and more confined to the core area. The maximum total hemoglobin concentration was reduced by about 10 μmol/liter and the average was about the same as before. This example demonstrates the feasibility of monitoring the treatment using the combined technique.

To correlate the near images with microvessel densities, three block samples obtained at breast-conservation surgery marked as anterior, lateral and posterior were selected (see FIG. 15) for microvessel counting. The total number of microvessels were 196 (lateral), 114 (anterior) and 48 (posterior and inferior) per 10 consecutive fields at a magnification of 200× respectively (see Table 1: Sample #19). The high counts obtained at anterior and lateral block samples correlate with the high optical absorption and total hemoglobin concentration shown in slice 3 of FIGS. 15(b)-(d). A representative section demonstrating high microvessel density is shown in FIG. 15(e). Immunohistochemical staining with antibody to Factor-VIII highlights the endothelial cells (stained brown) lining the vessels within the section. The low counts obtained at the posterior and inferior block samples correlate with the low optical absorption as well as the low hemoglobin concentration as seen in the deeper slices 4 and 5 of FIG. 15(a)-(c).

TABLE 1

| | | ^{@}mvd per | | |
|---|---|---|---|---|
| | | 10 fields at a | | ^{@}mvd per 10 fields at |
| Sample | | magnifica- | | a magnification of |
| # | location | tion of 200× | location | 200× |
| 19 | NA | NA | ***LAT | 196 |
| | NA | NA | ANT | 114 |
| | NA | NA | POST/^{##}INF | 48 |
| 5 | *ANT | 61 | ANT | 52 |
| | **POST | 40 | POST | 29 |
| 23 | NA | NA | POST/INF | 152 |
| | NA | NA | ANT/LAT | 60 |
| | NA | NA | ANT/^{#}MED | 88 |
| 21 | NA | NA | POST/LAT | 121 |
| | NA | NA | POST | 124 |
| | NA | NA | ANT | 83 |

*ANT: anterior;
**POST: posterior;
***LAT: lateral;
^{#}MED: medial;
^{##}INF: inferior
^{@}total microvessels per 10 consecutive fields at a magnification of 200×.

### Example 4

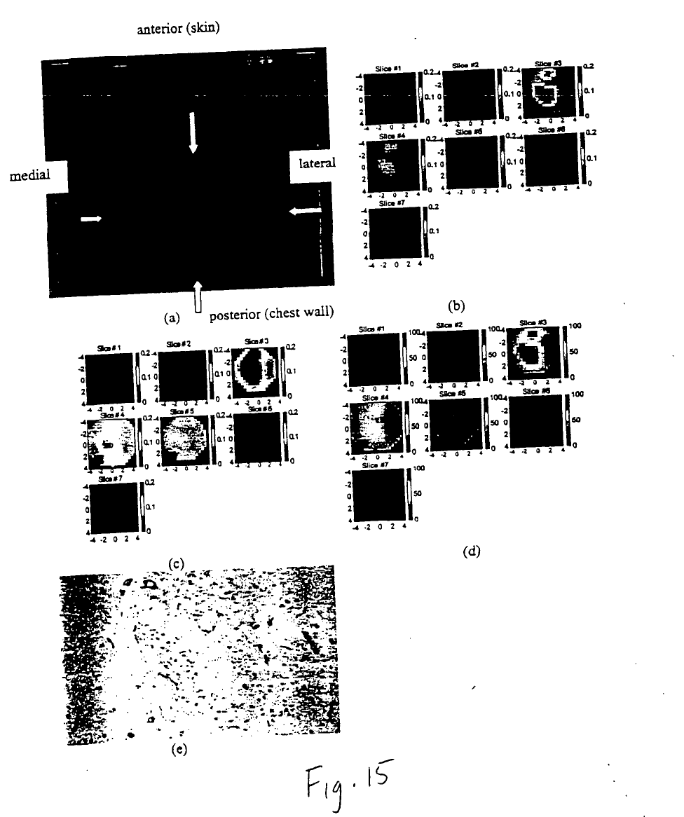

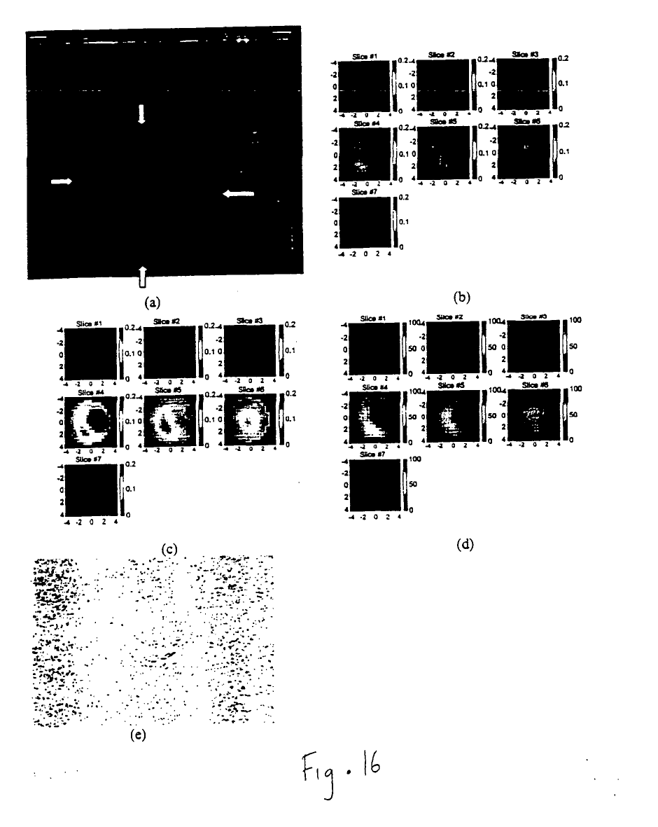

This imaging example was conducted on a 47-year-old woman who had a 3 cm×3 cm×2 cm dominant mass at the 2 o'clock position in her left breast. The lesion center was about 2.3 cm in depth relative to the skin. Ultrasound showed a hypoechoic mass with irregular margins as may be seen in FIG. 16(a), and the lesion was considered highly suspicious for malignancy. FIG. 16(b) and (c) are optical absorption maps obtained at 780 nm and 830 nm, respectively, and FIG. 16(d) represents the total hemoglobin distribution. Since the normal tissue boundaries on top and bottom of the cancer can be visualized well in ultrasound, the region of interest for finer grid optical reconstruction was chosen as 9 cm×9 cm>2 cm with larger spatial dimensions than the ultrasound measured ones, to account for spatial location uncertainties. The light absorption at both wavelengths was much lower than that in the previous example, but the distributions are highly heterogeneous. The measured maximum absorption coefficients at 780 nm and 830 nm are 0.08 cm⁻¹ and 0.10 cm⁻, respectively. The measured maximum total hemoglobin concentration of the tumor and average of the lesion were 40.6 μmol/liter and 17.2 μmol/liter, respectively. The surgical pathology report revealed that the mass was an infiltrating carcinoma (histological grade II, nuclear grade II) with low mitotic activity. The total counts of microvessels obtained from anterior and posterior core biopsy samples were 61 and 40 per 10 consecutive fields at a magnification of 200×, respectively as may be seen in Table 1, Sample #5. The total counts measured from anterior and posterior tumor samples obtained at definitive surgery were 52 and 29, respectively. These low counts correlate well with the low optical absorption shown in FIG. 16(b)-(c) and indicate that the tumor was poorly perfused. A representative section demonstrating low microvessel density is shown in FIG. 16(e).

Since the hand-held probe could be easily rotated or translated, at least three co-registered ultrasound and near infra-red data sets were obtained at the lesion location for the patient. Reconstructed corresponding optical absorption maps as well as the total hemoglobin concentration distribution under the co-registered ultrasound guidance were also obtained. The measurements shown in Table 2 are average values taken over three images with standard deviations given in parenthesis. The maximum absorption coefficient, the average absorption coefficient within the hot area defined as within 6 decibels (dB) of the maximum value and denoted as average μₐ, the ratio of average absorption coefficient inside the lesion calculated within the region of finer grid (ROI) used for near infra-red imaging reconstruction over the hot area, the maximum total hemoglobin concentration, the average total hemoglobin concentration inside the region of interest, are all given in Table 2. The ratio of average absorption coefficient inside the region of interest over the hot area partially reflects the angiogenesis heterogeneity.

TABLE 2

| | max μₐ cm⁻¹ | ave μₐ cm⁻¹ | ave ROI/ave μₐ | max μₐ cm⁻¹ | ave μₐ cm⁻¹ | ave ROI/ave μₐ | max total Hb | aveHB |
|---|---|---|---|---|---|---|---|---|
| Sample ID# | (780 nm) | (780 nm) | (780 nm) | (830 nm) | (830 nm) | (830 nm) | μmol/liter | μmol/lite |
| 19 | 0.16 (0.01) | 0.11 (0.01) | 33.7% | 0.18 (0.004) | 0.13 (0.006) | 50.2% | 80.6 (2.4) | 24.1 (1.1) |
| 5 | 0.08 (0.01) | 0.05 (0.01) | 56.7% | 0.10 (0.01) | 0.06 (0.01) | 67.4% | 44.0 (5.3) | 17.2 (1.1) |
| 23 | 0.16 (0.01) | 0.11 (0.01) | 21.1% | 0.33 (0.07) | 0.24 (0.05) | 29.8% | 113.0 (21.4) | 24.5 (2.4) |
| 21 | 0.20 (0.01) | 0.15 (0.01) | 16.7% | 0.29 (0.01) | 0.21 (0.01) | 30.5% | 114.8 (5.1) | 20.9 (0.7) |

max μ_{a:} measured maximum absorption coefficient.
ave μₐ: mean μₐ calculated within FWHM region from the maximum value.
ave ROI: mean μₐ value calculated inside ROI for finer grid NIR imaging reconstruction
max total Hb: maximum total hemoglobin concentration
aveHb: mean total hemoglobin concentration calculated within FWHM region from the maximum value.

### Example 5

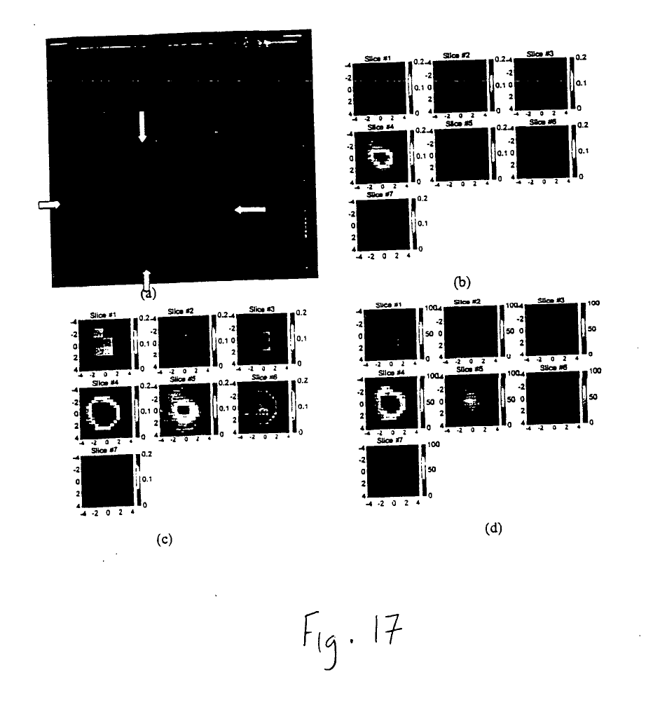

The third patient was a 33-year-old pregnant woman who had palpable left breast lump at 12'clock position measuring 3 cm×3 cm×1.5 cm. The ultrasound image showed that the lesion had discrete nodularity as may be seen in FIG. 17(a). The center of the lump was about 2.5 cm deep from the skin. An ultrasound guided core biopsy was obtained and revealed that the lesion was an invasive ductal carcinoma as seen on multiple cores. The lesion was categorized as a histologic grade III, nuclear grade III. The results are shown as sample #23 in Table 1. Optical absorption maps as well as a total hemoglobin concentration map were obtained 8 days after the core biopsy at the time of the patient visit as may be seen in FIGS. 17(b) and (c) respectively. The measured average maximum absorption coefficients at 780 nm and 830 nm are 0.15 cm⁻¹ and 0.33 cm⁻¹, respectively as may be seen in Table 2, sample #23. The maximum total hemoglobin concentration of the tumor is 113.0 μmol/liter and the average is 24.5 μmol/liter. The region of interest used for finer grid optical reconstruction is chosen as 7 cm×7 cm×1.7 cm. As seen from Table 2, the standard deviation of maximum total hemoglobin concentration measured from different probe positions is 21 μmol/liter, which is much larger than those obtained from other cases.

Histological microvessel counts of three sample blocks were 60 (anterior/lateral), 88 (anterior/medial), and 152 (posterior and inferior) per 10 consecutive fields at a magnification of 200× respectively. The larger variation in total counts at different tumor locations is partially related to the inherent heterogeneity of breast tumors and the resulting misdistribution of angiogenesis in the viable and schirrous regions. Nevertheless, the relatively higher counts obtained at anterior/lateral, anterior/medial sample blocks correlate to some extent with the high optical absorption and total hemoglobin distribution as seen in slice 4 of FIGS. 17(b)-(c). FIG. 17(d) represents the total hemoglobin distribution. The large number of counts obtained in the posterior and inferior sample blocks do not correlate with the low optical absorption distribution seen by optical imaging in deeper slices. Without being limited by theory, it is believed that for deeply located, highly absorbing tumors, such as those seen in the present Example and the following Example 6, the diffusively reflected photon density waves from the bottom of the tumor are weak when they reach the detectors. Therefore, the perturbations from the deeper part of the tumor are much weaker compared with perturbations from the top part of the tumor. As a result, the reconstructed images show higher absorption at the top part of the tumor and lower absorption at the bottom part of the tumor. For the poorly perfused case, the light absorption by the tumor was not high and the lesion was imaged more uniformly from top to bottom. Once again, without being limited by theory, it is believed that this depth dependent distribution imaged by diffused wave may be minimized by increasing the detection sensitivity and appropriately scaling the weight matrix for imaging reconstruction.

### Example 6

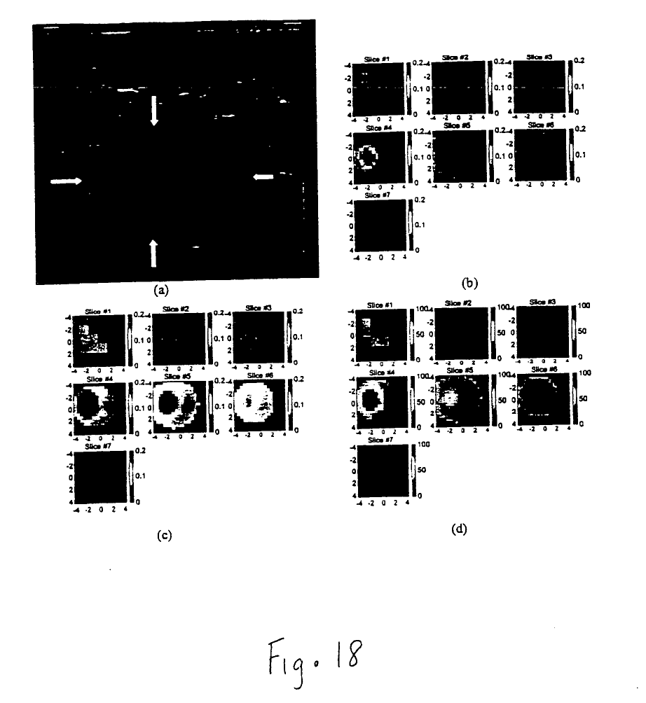

The last example was obtained from a 53-year old woman who had a palpable mass, but a normal—mammogram. An ultrasound obtained revealed an irregularly shaped lesion of 2 cm×2 cm×1.3 cm as may be seen in FIG. 18(a) and ultrasound guided surgical biopsy confirmed an invasive ductal carcinoma (histological grade II, nuclear grade II). The results are shown in Table 1 as Sample #21. The lesion center was about 2.5 cm in depth relative to the skin. Optical absorption maps as well as the total hemoglobin concentration distribution were obtained as shown in FIGS. 18(b)-(c). FIG. 18(d) represents the total hemoglobin distribution. The region of interest used for finer grid optical reconstruction was chosen as 8 cm×8 cm×1.6 cm. The measured maximum absorption coefficients at 780 nm and 830 nm were 0.20 cm⁻¹ and 0.29 cm⁻¹, respectively. The maximum total hemoglobin concentration in the tumor was 114.8 μmol/liter and the average was 20.9 μmol/liter. The total number of microvessels were 83 (anterior), 121 (posterior and lateral), 124 (posterior) per 10 consecutive fields at a magnification of 200×, respectively. The higher anterior and lateral counts correlate with the high optical absorption and high total hemoglobin concentration. Similar to the patient in Example 5, the higher posterior counts do not correlate with the low light absorption and low hemoglobin concentration seen in deeper slices. The reason for the lack of correlation is explained in the above example.

The present invention can be embodied in the form of computer-implemented processes and apparatuses for practicing those processes. The present invention can also be embodied in the form of computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. The present invention can also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

The use of ultrasound in conjunction with near infrared diffusive light can overcome some of the difficulties associated with utilizing ultrasound in conjunction with mammography. For example, the overlapping appearances of benign and malignant lesions makes ultrasound less useful in differentiating solid lesions resulting in a large number of benign biopsies. However, combining diffused light imaging in the near infrared region with ultrasound provides a novel way for the detection and diagnosis of solid lesions.

While the invention has been described with reference to a preferred embodiment and various alternative embodiments, it will be understood by those skilled in the art that changes may be made and equivalents may be substituted for elements thereof without departing from the scope of invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A method for imaging a lesion using combined near infrared diffusive light and ultrasound, the method comprising:
scanning a subject with ultrasound waves to obtain ultrasound images of a scanned volume, the scanned volume including the lesion;
scanning the subject with near infrared light to obtain optical measurements of the scanned volume;
segmenting the scanned volume into a lesion region including the lesion and a background region absent the lesion using the ultrasound images; and
reconstructing from the optical measurements an optical image of at least a portion of the scanned volume, the reconstructing being performed using different voxel sizes for optical measurements corresponding to the lesion region and optical measurements corresponding to the background region.

2. The method of claim 1, further comprising:
measuring parameters of the lesion using the ultrasound images to provide values indicative of the parameters; and
reconstructing the optical image again using the values.

3. The method of claim 1, wherein the optical measurements include amplitude and phase.

4. The method of claim 1, wherein the reconstructing includes:
determining absorption and scattering coefficients at slice depths in the scanned volume.

5. The method of claim 1, wherein the optical image indicates at least one of wavelength-dependent absorption associated with the lesion and hemoglobin concentration associated with the lesion.

6. The method of claim 2, wherein the values indicate lesion location in the scanned volume and size of the lesion.

7. The method of claim 6, wherein the reconstructing the optical image again includes:
increasing a value indicating lesion size to account for possible inaccuracies of an initial lesion size estimate.

8. The method of claim 7, wherein the value indicating lesion size is a value indicating a diameter of the lesion.

9. The method of claim 7, wherein the reconstructing the optical image again further includes:
controlling the total number of voxel sizes.

10. A method for imaging a lesion using combined near infrared diffusive light and ultrasound, the method comprising:
scanning a subject with ultrasound waves to obtain ultrasound images of a scanned volume, the scanned volume including the lesion;
scanning the subject with near infrared light to obtain optical measurements of the scanned volume;
segmenting the scanned volume into a lesion region including the lesion and a background region absent the lesion using the ultrasound images;
reconstructing from the optical measurements an optical image of at least a portion of the scanned volume;
measuring parameters of the lesion using the ultrasound images to provide values indicative of the parameters; and
reconstructing the optical image again using the values.

11. The method of claim 10 wherein the reconstructing from the optical measurements is performed using different voxel sizes for optical measurements corresponding to the lesion region and optical measurements corresponding to the background region.

12. The method of claim 10, wherein the optical measurements include amplitude and phase.

13. The method of claim 10, wherein the reconstructing includes:
determining absorption and scattering coefficients at slice depths in the scanned volume.

14. The method of claim 10, wherein the optical image indicates at least one of wavelength-dependent absorption associated with the lesion and hemoglobin concentration associated with the lesion.

15. The method of claim 10, wherein the values indicate lesion location in the scanned volume and size of the lesion.

16. The method of claim 15, wherein the reconstructing the optical image again includes:
increasing a value indicating lesion size in the scanned volume to account for possible inaccuracies in an initial estimate.

17. The method of claim 16, wherein the value indicating lesion size is a value indicating lesion diameter.
